(19) United States
(12) Patent Application Publication
(10) Pub. No.: US 2005/0070010 A1
(43) Pub. Date: Mar. 31, 2005

(54) Dockable processing module

(76) Inventors: Thomas Laurell, Lund (SE); Johan Nilsson, Bjarred (SE); Gyorgy Marko-Varga, Malmo (SE); Simon Ekstrom, Lund (SE); Lars Wallman, Sjobo (SE)

Correspondence Address: LADAS & PARRY LLP, 224 SOUTH MICHIGAN AVENUE, SUITE 1200, CHICAGO, IL, 60604, US

(21) Appl. No.: 10/497,996

(22) PCT Filed: Dec. 11, 2002
(86) PCT No.: PCT/SE02/02285

(30) Foreign Application Priority Data
Dec. 11, 2001 (SE) 0104125-0
Jul. 15, 2002 (SE) 0202222-6
Aug. 13, 2002 (SE) 0202415-6

(51) Int. Cl. C12M 1/34

(52) U.S. Cl. 435/287.2

(57) ABSTRACT

A processing module for extracting certain biomolecules from a solution, comprising an extraction unit having at least one elongated channel (101) each of said at least sine channel have un inlet (102) and an outlet (103) and being provided with adhering units (201), each said unit being provided with adhesive means, having affinity for said certain biomolecules, said extraction unit further comprises docking means (205) having an inlet array and an outlet array, that enables the extractor to be docked to and undocked from other devices having corresponding docking means, such that said solution or another fluid can be made to flow from said other devices, entering the inlet array, to flow through the at least one channels(101) and to leave the extraction device via the outlet array.

### FIELD OF INVENTION

The present invention relates to methods and devices for chemical analysis. More specifically it relates to devices for processing biological specimens. Yet more specifically it relates to devices for extracting biomolecules, for example peptides and/or proteins, from a mixture of molecules in a solution.

### BACKGROUND

Chemical analysis and particularly biomolecular analysis of e.g. proteins in a biological specimen are experiencing an increased demand for speed and accuracy. Different techniques for increasing separation, extraction and preparation of different parts of a specimen have being suggested, but there is still a problem to really increase handling and processing times for large multiple specimens investigations.

WO0138865A1 to Harrison et al., discloses an apparatus and method for trapping bead based reagents within microfluidic analysis systems.

U.S. Pat. No. 6,265,715 to Perreault et al., discloses a non-porous membrane for MALDITOF MS, claiming a method comprising the following steps: providing a non-porous membrane as a sample support; providing a matrix solution; applying the analyte sample directly to the non-porous membrane; allowing the analyte sample to dry; applying the matrix solution to the dried analyte sample; allowing the matrix solution to dry; mounting the non-porous membrane onto a probe body; inserting the probe body and the non-porous membrae into a mass spectrometer; and carrying out MALDI-TOFMS analysis of the analyte sample.

U.S. Pat No. 6,074,725 to Kennedy, (Caliper) discloses a method for fabrication of microfluidic circuits by printing techniques including providing laminates having microfluidic structures disposed between sheets of the laminate.

U.S. Pat. No. 2002/0,039,751 A1 discloses high throughput screening assay systems in microscale fluidic devices.

EP1163052A1, to Burd Mehta et al., identical to WO0050172 discloses manipulation of microparticles in microfluidic systems.

U.S. Pat. No. 5,969,353 to Hsieh discloses a microfluid chip mass spectrometer interface comprising a very fine tube to an outlet port of a microfluid chip, enhancing the sensitivity of mass spectroscopy analysis of materials exiting the outlet port.

WO 0046594 to Dubrow et al., (EuroPCT EP1159605A1) discloses methods, devices and systems for characterizing proteins.

U.S. Pat. No. 5,646,048 discloses an analytical apparatus having a microcolumn and an interface system for controlling the flow from a first microcolumn to a second microcolumn.

WO 01/56771 discloses a manufacturing method for micro structures having different surface properties in a multilayer body using plasma etching.

WO 99/22228 discloses a multichannel system for separation, collection and analysis of samples. The device makes use of a solution permeable gel and capillary columns for separation.

U.S. Pat. No. 4,908,112 discloses a silicon semi-conductor plate (wafer) for analysing biological specimens of micrometer size. Channels sealed with glass plates is arranged together with electrodes to activate fluid motion through the channels using electroosmosis.

U.S. Pat. No. 3,915,652 discloses a transport system for analytical specimens using capillaries sealed between movable nozzles.

U.S. Pat. No. 5,595,653 discloses a micro column for extraction of assays from liquids, comprising an extraction media having a particle size less than 20 microns that is held on place and compressed by two compression layers.

U.S. Pat. No. 5,965,237 discloses a microstructure device comprising a support element and a flat surface and a micro structure element having a microstructure surface with both even surface components and recesses. Material is poly (dimethylsiloxane) glass, silicon, or the like.

U.S. Pat. No. 4,891,120 discloses a chromatographic separation device comprising a body of semiconductor material and having a channel arranged in the surface layer to house liquid or solid phase material for a chromatological test or separation procedure. The channel comprises at least one electrode and may be provided with an electronic or optical system.

Important for all biochemical analysis systems is to keep the dispersion to a minimum. When dealing with detection of low concentration analytes it is also of importance to keep the area of the surfaces in e.g. interconnecting tubings/channels to a minimum in order to avoid unspecific analyte adsorption.

If bead based techniqes are used it is of importance to simplify the loading and unloading of the beads and analytes. Using integrated systems, i.e. the bead trapping unit is integrated in the analysis system, as in WO0138865A1 (Harrison) or EP1163052A1 (Burd) requires special arrangements for the handling of the beads which will reduce the overall throughput.

### SUMMARY

This invention satiesfies the above mentioned need for increased handling speed of biological specimens. In particular it increases handling speeds for such specimens subjected to analysis involving separation of the specimen into different fractions where each fraction is subjected to subsequent extraction of analytes. Embodiments of the invention also greatly simplifies the loading and unloading of beads in bead based systems.

A typical embodiment of the invention comprises an extraction device for extracting certain biomolecules from a solution, comprising at least one elongated channel for passage of the specimen in fluid phase, said channel having an inlet end and an outlet end and being provided with an adhering unit for the capture of certain biomolecules, each said unit being provided with adhesive means, having affinity for said certain biomolecules. The extraction device further comprises docking means having an array of inlet openings and an array of outlet openings, that enables the extractor to be docked to and undocked from other devices having corresponding docking means, such that said specimen or another fluid can be made to enter through the docking means inlet openings, flow through the at least one channels of the device and to leave the extraction device via the docking means outlet openings.

The increased handling speed is achieved when having multiple extraction devices capable of being handled in a pipeline or assembly line fashion. In a typical embodiment one extraction device is docked to a priming device where it is primed with adhesive means, e.g. microbeads with a surface coating of adhesive molecules having affinity to the molecules that are to be extracted. The extractor is subsequently undocked from the priming device and docked into a specimen loading device, where a specimen or preferably a number of fractions of a specimen in fluid phase is loaded, via said docking means, into the channels of the extractor, enabling certain molecules to adhere to the microbeads. Subsequent to said loading of the extractor, said extractor is undocked from the specimen loading device and docked to a washing device that flushes the extractor channels with a washing solution via the docking means. The flow of fluid is kept in the same direction all the time, i.e. from inlet to outlet. The extractor can then be undocked from the washing device. The extractor can now be stored away for some time if this is desirable. In most cases, however, the extractor is docked without delay to an elution device where an eluant is provided to flow through the channels of the extractor and eluate the certain molecules from the microbeads, forming separate eluates passing out from the outlet openings of the docking means. The eluates can then be collected for an immediately following analysis or for further processing. Further processing may include micro dispensing (e.g. piezo electric micro dispensing) of at least parts of said eluates on a target plate suitable for subsequent MALDI-TOF mass spectrometry.

### FIGURES

Embodiments of the invention is disclosed in the following description and discribed with the aid of the following figures in which

FIG. 1a shows a combined device comprising a separator, an extractor array according to an embodiment of the invention and a dispenser array

FIG. 1b shows in cross section the dispenser array and the beneath arranged target plate

FIGS. 2a, b and c shows a dockable extractor according to an embodiment of the invention.

FIG. 3 illustrates the process of separating (1), extracting (2), washing(3), eluating and dispensing (4)

FIG. 4 shows an alternative embodiment of a dockable extractor and how it is docked

FIGS. 5a and b shows an alternative embodiment of the combined extractor of FIG. 1.

FIG. 6 shows a dockable extractor (extractor cartridge) comprising multiple extractors and bending notches.

FIG. 7a shows a view from above of an embodiment of the dockable microextraction chip of the “2D-Array” type, together with a cross section of the same.

FIG. 7b shows a view from above of an embodiment of the dockable microcxtraction chip of the “Film-strip” type.

FIG. 8 shows a view from above of an embodiment of the dockable microextraction chip arranged at a circular disc, together with a detail.

FIG. 9 shows angular views illustrating the steps of using film-strip and circular embodiments for loading, extracting, and eluating/dispensing samples.

FIG. 10a shows a side cross section of an embodiment having a droplet inlet zone

FIG. 10b shows a view from above of the embodiment in FIG. 10a

FIG. 11 shows a schematic cross section of an electrospray nozzle and power source.

FIG. 12a shows components for performing sample loading, washing, docking, and extracting

FIG. 13 shows principal robotic steps for an embodiment of a method and a device according to the invention using the components of FIG. 12.

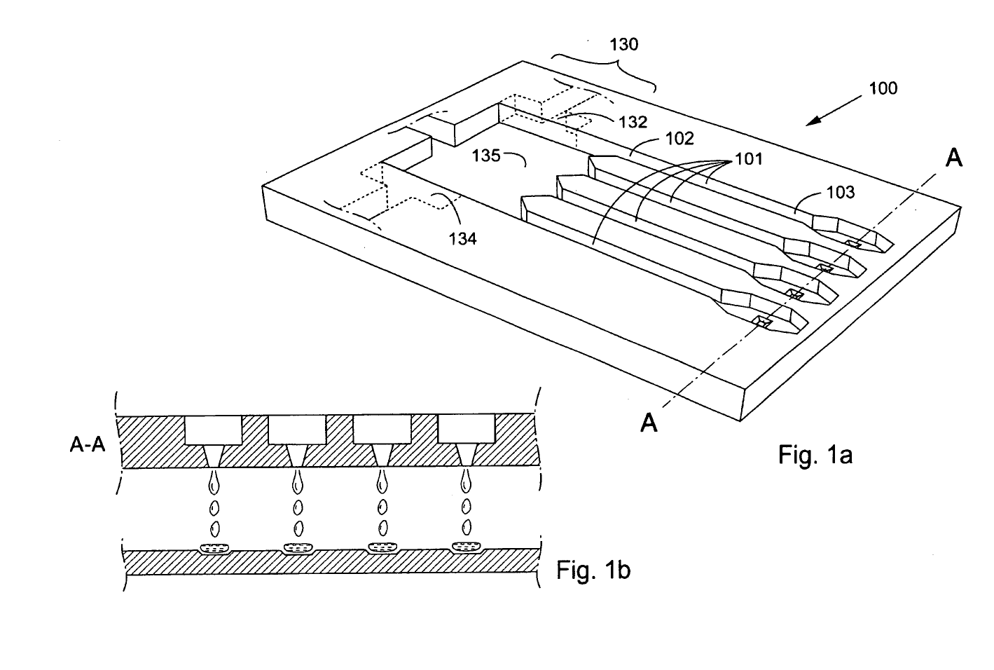

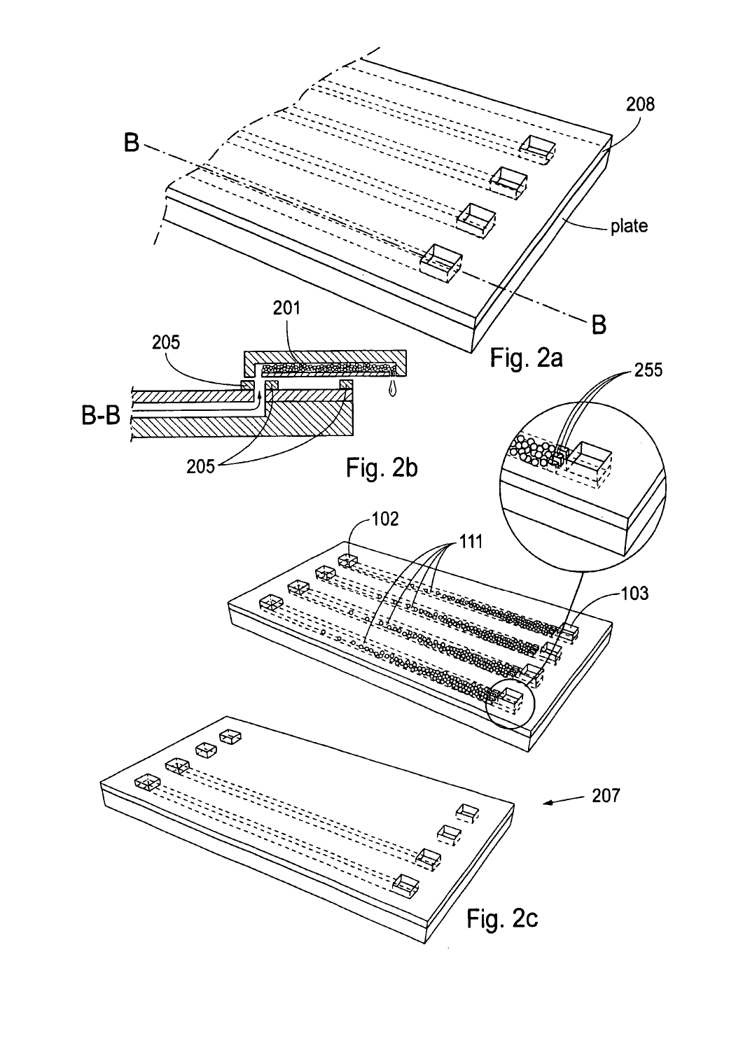

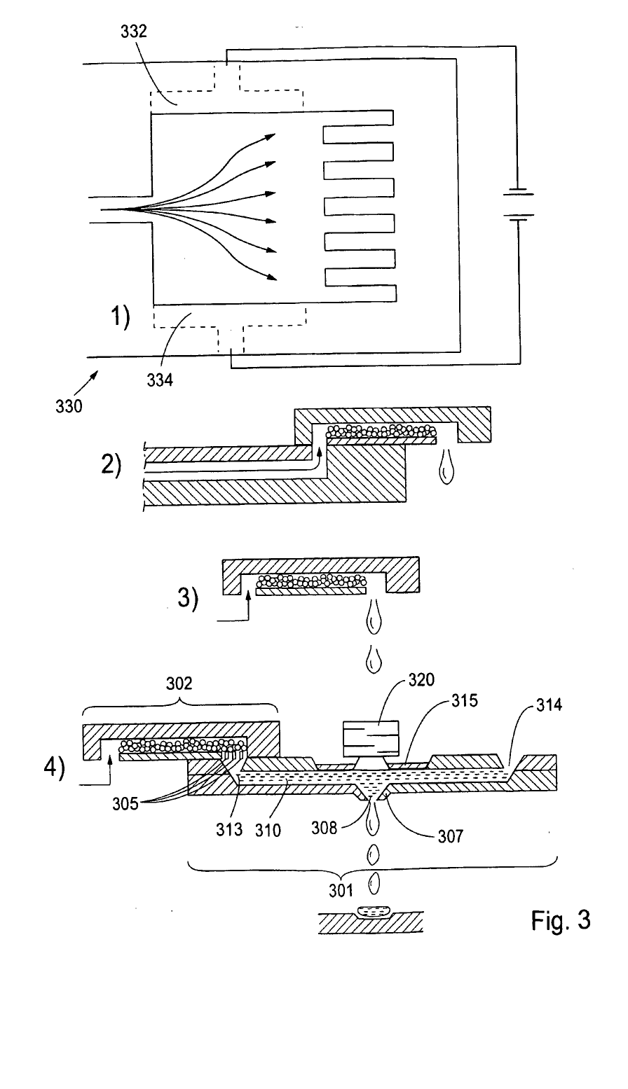

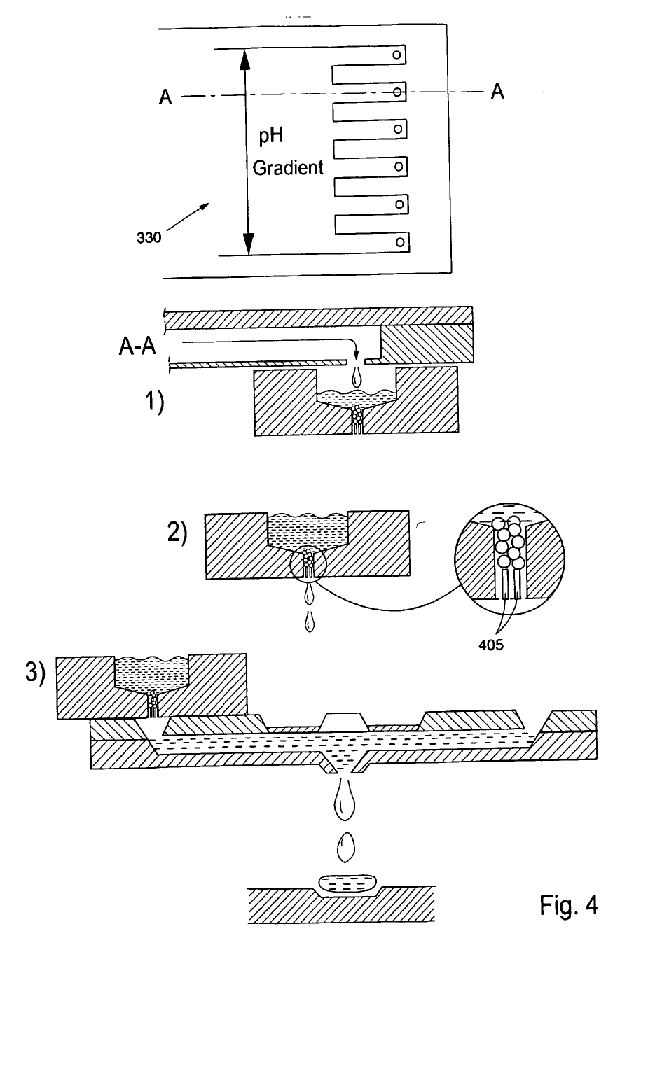

### DESCRIPTION

In this description the term “virtual flow channel” is intended to mean a microscopic flowing portion of a laminary flowing fluid, said portion having a long axis being parallel to the direction of flow, and said portion having a width and a depth orthogonally to the direction of flow, said portion can be regarded as an entity not mixing with the rest of the flowing fluid because of said laminar flow and small (micro) dimensions, thus constituting a “virtual channel”. Alternative term: “virtual channel flow”, “virtual flow line” and “virtual flow lane”.

The inventive concept of the present invention lies in a dockable and disposable processing module comprising a micro-extractor arranged to facilitate extraction, enrichment, and eluation of certain analyte biomolecules origination from a sample solution.

Extractor

A first embodiment of the invention comprises an extractor having a number of separate channels 101, 111 each devised to contain a porous bed 201, able to adsorb species from the one of the components of a mixture that is brought to pass through it. Said bed can comprise e.g. a bed of microscopic beads. The channels 101, 111 are arranged having microscopic dimensions. The width of a channel is typically less than a few tenth of a millimeter, often even smaller. The depth of a channel is in this magnitude too. The microscopic beads are prevented from escaping from the channels by a restraining means 255, 305, 405. Said restraining means can comprise a mesh, or a number of columns arranged having interspaces smaller than the diameter of the micro beads.

As an alternative, the porous bed can be omitted and the function to adsorb species to be analysed can be carried out by means of modified surfaces forming part of the walls that define the channels. To increase the efficiency the surfaces may be subject to a surface enlarging treatment e.g. forming of a porous layer. Embodiments include surfaces comprising surface modified silicon and porous silicon.

In a methodological step said species is eluted by the aid of an eluant forming an eluate corresponding each component, i.e. a type of solid phase extraction, SPE.

Dockable Extractor

In an alternative embodiment the extractor is designed to be dockable. With this term is meant that said extractor is attachable to, detachable from and re-attachable to other devices. Said dockable extractor 207 comprises a plate or another movable entity that is devised to be manually or automatically detachable from other parts of e.g. an analysis device. Said extractor is also devised to be re-attachable to the same or other parts of the analysis device, such as a washing device or a dispensing device. Specifically such embodiments comprise docking means that enables the docking and the flow of liquid from other parts of the analysis device to the inlets of the extractor, and the flow of liquid from outlets of the extractor to other parts of the analysis device. Such parts may include a feeding device or a washing device, or an elution device, or a combination thereof. Said docking means can also comprise means for preventing species from escaping from the dockable extractor, despite of mechanical handling. Said means can comprise the arranged small dimensions, that will keep the species in the extractor by the aid of capillary forces.

In a preferred embodiment the extractor part of the docking means comprises a flat surface with a number of holes, each hole being provided with a sealant mechanism slightly protruding from the surface. The sealant mechanism may be formed by patterning a polymer using lithographic technique. In an alternative embodiment the sealant mechanism comprises a hydrophobic break formed using surface modifying technology. In further alternative embodiments the hydrophobic break is achieved by arranging a polymer film surrounding the hole. Still other embodiments comprise sealant layers comprising miniature gaskets or o-rings.

The docking means also comprises a fastening system of notches and protruding parts keeping the dockable extractor in determined position so that the holes of the extractor part of the docking means connect to and align with the corresponding holes of the part it is docked to. The fastening system also exerts a certain mechanical pressure to assure tightness of the connection. The fastening system is also devised to enable appropriate attachment and detachment of the dockable extractor.

Droplet Capillary Loading, Filter Paper Drainage

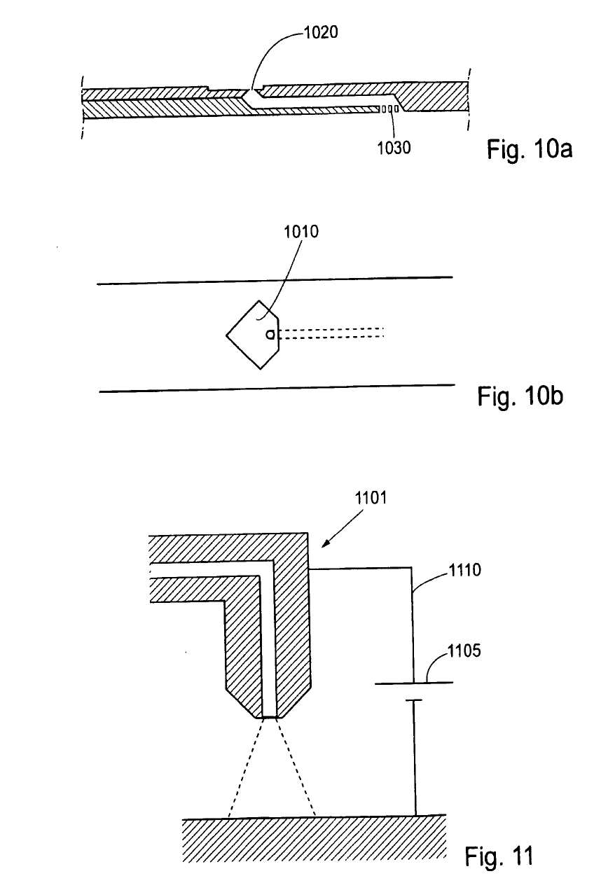

Referring to FIG. 10, supplying analyte solution to an extractor according to an embodiment of the invention, is performed by pipetting a droplet of said solution in a droplet inlet zone 1010, said zone having a direct fluid connection 1020 to the extractor bed. Capillary forces will subsequently fill the extractor because of the small dimensions of the channels of the extractor. Fluid is then drained through the extractor by applying a filter paper at the outlet 1030, said paper having suitable capillary characteristics for draining all the fluid through the extractor, leaving no remains of the droplet at the droplet inlet zone or any greater amounts of fluid inside the extractor. The same procedure of droplet loading and filter paper drainage can be used for washing and elution.

Typically a droplet of 50 microlitres is pipetted in a droplet inlet zone 3 by 3 millimetres and 300 micrometres deep.

Multiple Microextractor Assemblies

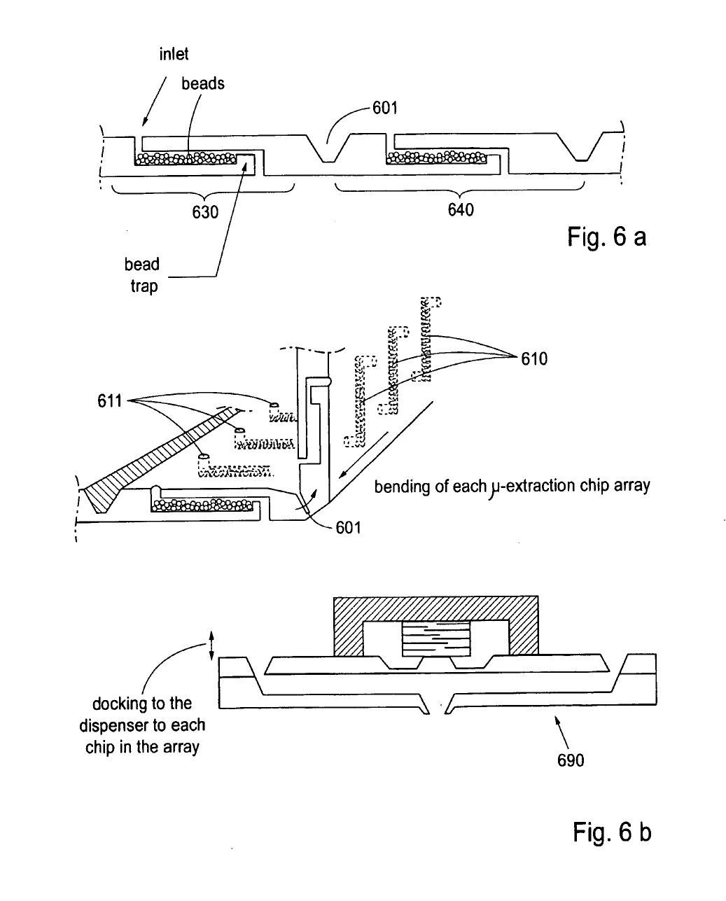

In alternative embodiments of the dockable extractor, see FIGS. 6, 7, 8, and 9, an extractor assembly is devised that comprises a multitude of extractor arrays providing for “assembly line” efficient and roboted fast handling of extractor modules:

Straight Linked Chain

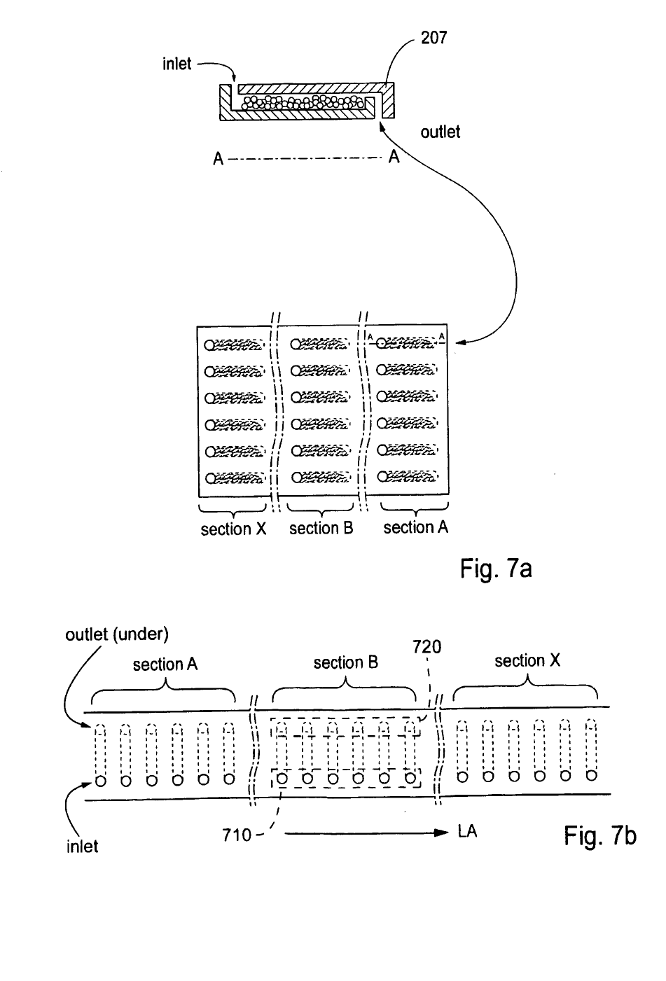

In one of these alternative embodiments of the dockable extractor, see FIG. 6 and FIG. 7a, a linked chain extractor-assembly comprises a multitude of extractor arrays 630, 640 etc parallel to a long axis of the chain and orthogonally running notches 601 separating one extractor array from another. Each extractor array comprises a number of extractor channels 610. By bending the cartridge at a certain notch one of the separate arrays are enabled to dock to e.g. a following dispenser array 690, because the preceding extractor arrays is bent upwards, leaving space available for the next extractor array. Bending at another notch enables another array to dock to said dispenser array 690.

Orthogonally Linked Chain (Film-Strip)

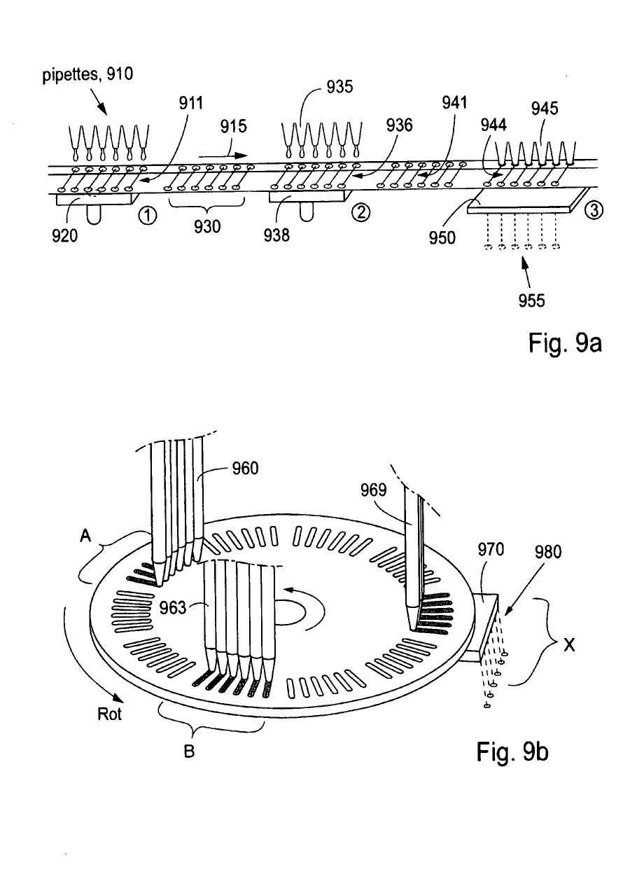

In FIG. 7b is shown an orthogonally linked chain extractor-assembly comprising a multitude of extractor arrays, also called sections, orthogonally running compared to a long axis LA of the chain. Said orthogonally linked chain provides docking surfaces 710, 720 at the long sides of the chain, providing for easy access to many microextraction arrays simultaneously. In FIG. 9a is shown how pipettes 910 are arranged to supply analytes to extraction arrays 930 having extraction beds 911, said extraction arrays 930 being devised to move along a type of “assembly line” handling device, implementing a processing method as described below. At a first location, in a first step a first set of pipettes 910 places droplets of analyte to inlets of extraction beds 911. Excess analyte is removed by a first suction device 920 arranged at extraction bed 911 outlets. Extraction array 930 is then moved forward to a second location where a second set of pipettes 935 adds washing fluid to the extraction array and excess fluid is removed by a second suction device 938. Extraction bed is then moved forward to a third location where a third set of pipettes is supplying an eluation fluid to the extraction array and where a dispensing array 950 connected to the extraction array outlets collects and ejects the so eluted eluate as droplets 955.

Disk Unit (Circular Arrangement)

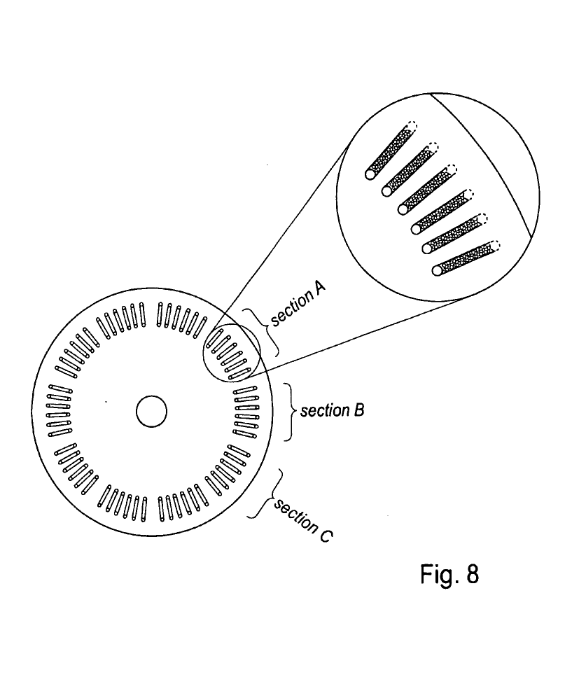

In FIG. 8 is shown another advantageous embodiment of a microextractor assembly comprising a circular disc or “daisy-wheel” arrangement where a number of sections A, B, C etc each comprises a microextraction array to be positioned/docked to either a pipetting/filter paper device for loading and draining the microextraction array as described above, or docked to another type of loading/draining device, as outlined in FIG. 9. In FIG. 9b is analogous to FIG. 9a shown a first 960, a second 963, and a third 969 set of pipettes having the same function as the corresponding set of pipettes 910, 935, 945, in FIG. 9a. A dispenser 970 ejects droplets 980 in a corresponding way as described above.

Storage Function

The embodiments of the microextractor described above can also, with no, or just minor modification be used as a storage unit, capable of retaining protein samples on the dockable microchip for long term storing e.g. at minus 20 degrees Celcius.

Dispenser

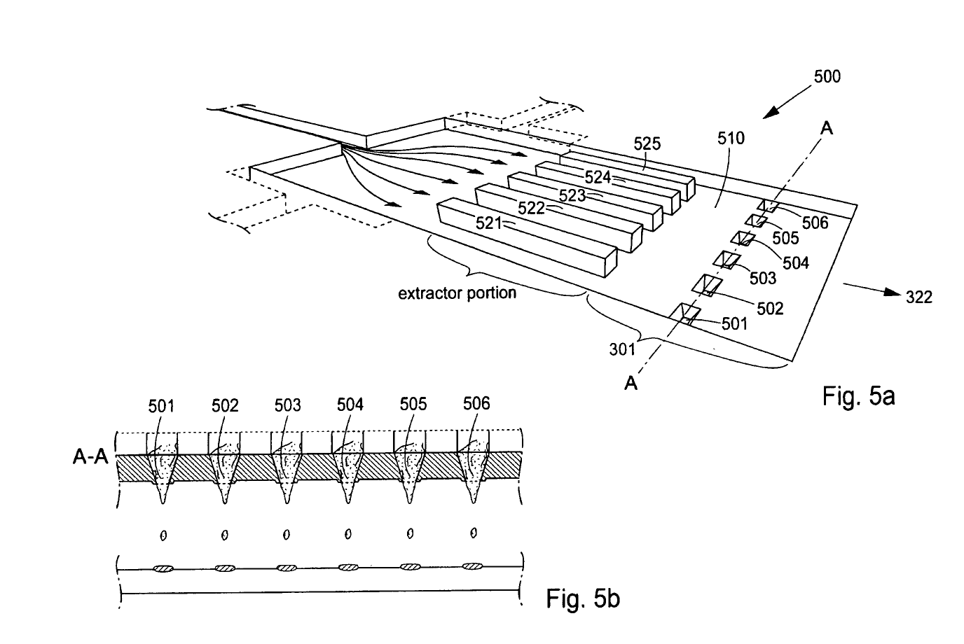

In another preferred embodiment, a processing module comprises an extractor portion 302 with functionality as described above and a dispenser portion 301. A first portion of the module comprises the extractor and a second portion, totally integrated with the first one, comprises an array of dispenser nozzle openings 501-506, (seen from “above” in FIG. 5a). Each separate flow of eluate is conducted to a separate dispenser nozzle. Said nozzles 501-506 can be arranged beside each other. Said nozzles can also be arranged in a zigzag or slightly displaced in relation to each other. Said nozzles are thereby forming a dispenser nozzle array.

In an alternative embodiment the separate flows of eluate is passing through a common basin 510 where the separating walls 521, 525, separating the different fractions is omitted downstream the restraining means 255 (not shown in FIG. 5), upstream of, but also near and at the dispenser nozzles 501-506. Said fractions are held separated in different laminar flow portions of the flowing liquid due to an arranged speed of said flow, and due the fact that the defining surfaces are devised to promote laminar flow. The speed of the flow is controlled by flow control means. The diffusion of the molecular species is kept at a minimum because of the relatively short time period/length during/under which the liquid has to flow when not guided by separation walls/surfaces.

In another embodiment the dispenser 301 comprises outlets or an outlet 322 enabling the fluid to flow through the dispenser without having to be dispensed through the dispenser nozzle. This facilitates priming and washing of the device.

Electrospray

Referring to FIG. 11 an alternative embodiment of the invention comprises electrospray nozzles 1101 and corresponding electrical power source 1105 and circuitry 1110 instead of piezoelectric actuators and dispenser nozzles, making said dockable extraction chip compatible with tandem mass spectrometry using electrospray; or other type of ionisation.

Isoelectric Focusing Means

In alternative embodiments the module is provided with isoelectric focusing means integrated together with the above described extraction means.

Said focusing means comprises a pair of electrodes 132, 134, 332, 334 integrated in the walls of a isoelectric focusing compartment 135 in the isoelectric focusing portion 130 of the module 100. Alternatively they are arranged within side compartments to the focusing compartment 135, said side compartments standing in fluid connection to said isolelectric focusing compartment 135, to reduce or inhibit gas production.

Material

The device is preferably manufactured in polymer or silicon. A master for mass production of polymer devices is preferably made from metal or from a ceramic material. Silicon is essentially inert when dealing with protein mixtures at room- or near room temperature. The material is also very suitable for micro-machining techniques, e.g. for etching away parts of the material with established etching techniques.

Another advantage using silicon, is that with said etching techniques the dimensions becomes very precise and it is possible to etch surface with far better than micrometer precision.

Structure

The device is preferably manufactured in a plate structure, where said channels are formed in a surface layer of a first plate. Said channels are subsequently sealed by bonding a second plate to the first plate.

Method

The above disclosed extraction device is used in a method for processing biological specimens with increased speed comprising the following steps; docking the extractor to a priming device for loading microbeads into the extractor and flushing the extractor with a priming solution, undocking the extractor from the priming device, flowing a biological specimen in fluid phase through the dockable microextraction device, letting certain biomolecules adhere to said microbeads inside said extractor, docking the extractor to a washing device, flushing the extractor, undocking the extractor from the washing device, docking the extractor to an elution device, eluting the certain biomolecules from the extractor. The biomolecules can be eluted directly to a dispensing device for being dispensed on a target plate for further processing using MALDI-TOF MS.

In a preferred embodiment a dispensing devision is arranged as a part of the extraction device, and in the corresponding method there is no need to dock the device to a special dispensing device, as would be realised by those skilled in the art.

Processing Steps

A preferred embodiment of a method according to the present invention comprises the following steps:
Docking a microextraction device/unit to a first process station (optional). Steps performed in such a first process station may comprise:
   Loading beads into the microchip, a so called packing step, where said beads form a microextraction bed; A slurry comprising an organic solvent/aqueous mixture in which particles are dissolved is supplied using high pressure (approx. one bar) into the dockable chip, thereby packing the beads/the slurry. This is of importance in order to obtain a high efficiency operating microextraction bed.

Preferably, in a second process station, the following steps are performed:
Activating the beads by alternatively applying an organic modifier/aqueous mixture, or applying an acidic aqueous solution.
Loading samples on the microextraction bed (microbeads), i.e., supplying sample at inlet either providing a pressure at inlet or by providing suction/low pressure at outlet or using capillary forces e.g by applying droplet to inlet and filter paper to outlet. After this step, samples are present in the chip purified/enriched 100-fold.
Washing the microextraction bed with a washing solution, e.g., a weak acid solution and/or a weak solvent either by using a pushing pressure at the inlet or a suction with low pressure at the outlet.
Drying the bed/beads e.g by supplying dry air through the channels.
Undocking the microextraction unit from the first process station (if needed)
(optional) Docking the microextraction unit to a third process station, and preferably to a handling device located downstream the microextraction unit, preferably a micro dispenser, either a single dispenser that sequentially is docked to each microextraction bed in each section, or an array of dispensers that matches (dock simultaneously) to all the microextraction beds in a section, or an integrated array dispenser that is docked to a whole section with microextraction beds, each corresponding to an ejecting nozzle in the array dispenser, said second process station being capable of eluating and dispensing/ejecting droplets now having a high concentration of desired analyte.
Eluting sample from beads
Dispensing sample onto target plate
Performing analytical read-out of the target plate
Dispose micro extraction module/micro extraction cartridge.

In this context it is possible to use the device to perform both global expression studies and focussed expression studies.

Robotic Components

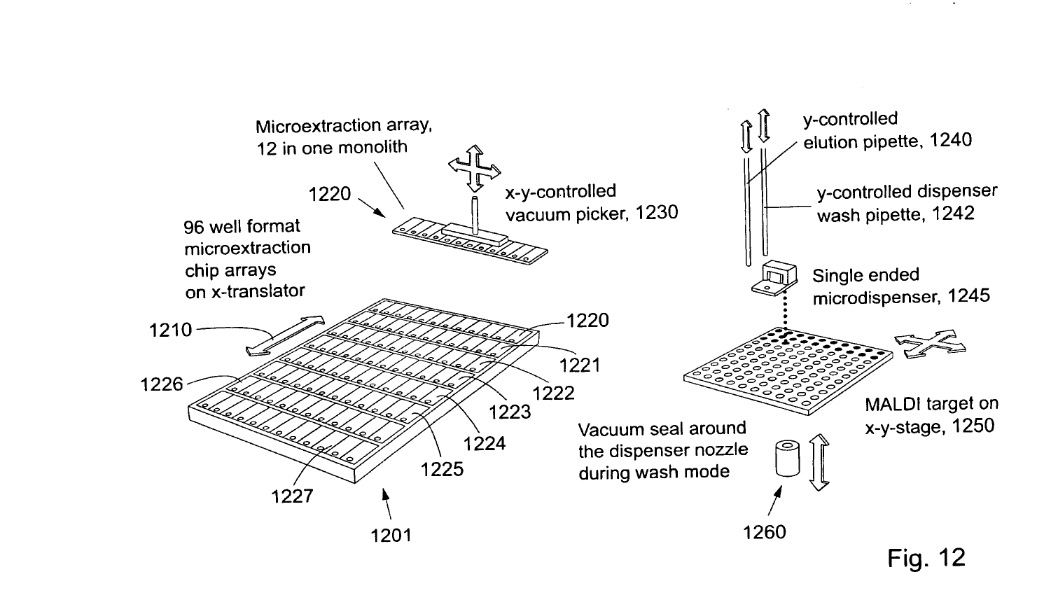

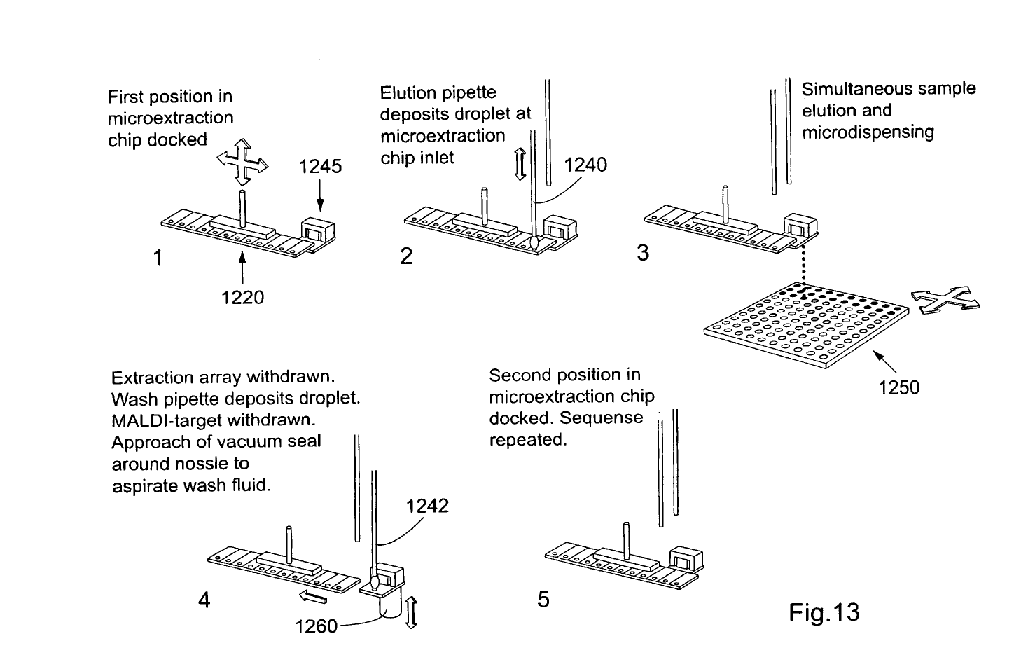

In FIGS. 12 and 13 is shown components for performing sample loading, washing, docking, and extracting together with the principal robotic steps for an embodiment of a method and device according to the invention. FIG. 12 shows a 96-well format microextraction chip array on a x-translator stage 1201. Said chip array is moveable in the direction indicated by the arrow 1210, henceforth referred to as the x-direction, by means of a x-translation device, or x-translator, not shown. The x-translator positions the chip arrays 1220-1227 so that they end up under a vacuum picker 1230. The arrays each includes twelve micro-extractor units, and is lifted and moved by an x-y, or z-y controlled vacuum picker 1230 arranged handle such microextraction array 1220-1227 one at a time. To the right in FIG. 12 is seen a y-controlled elution pipette 1240, and a y-controlled dispenser wash pipette 1242. As an alternative these pipettes can be z-controlled. The pipettes 1240, 1242 is arranged to be able to apply fluid, i.e. eluant and wash fluid, to the inlet opening of a single ended microdispenser 1245. The microdispenser 1245 is arranged so as to be able to dispense, in an ejective fashion, microscopic droplets towards a MALDI target 1250 on a x-y-stage. During washing a vacuum seal 1260 is applied around the dispenser nozzle.

FIG. 13 shows the principal robotic steps for (1) docking the microextraction chip 1220 to the microdispenser 1245, and (2) the subsequent sample elution, and (3) dispensing, and (4) removal of extraction chip and dispenser washing. Note that elution pipette 1240 is arranged to deposit droplets at microextraction chip 1220 inlet and that the wash pipette 1242 is arranged to deposit wash fluid droplets at the microdispenser inlet. During the wash operation the extraction array 1220 is withdrawn, the MALDI-target is withdrawn and the vacuum seal 1260 is approached around the microdispenser nozzle to aspirate wash fluid. When washing is completed the next position in the microextraction chip is docked and the sequence is repeated.

1. A processing module for extracting certain molecules from a solution, comprising a plate having at least two elongated channels, where each of said channels comprises a trench in said plate each trench is separated from nearby trenches by means of a dividing wall; said plate also comprises a sealing layer serving as sealant of said trenches;
each channel has an inlet and an outlet and is, provided with an adhering unit, each said adhering unit comprising adhesive means, having affinity for said certain molecules, said plate further comprises docking means, and at least one inlet and at least one outlet having fluid communication with said channels, that enables the extractor to be docked to and undocked from other devices having corresponding docking means, such that said solution or another fluid can be made to flow from said other devices, entering the inlets, to flow through the channels and to leave the extraction device via the outlets.

2. The module as recited in claim 1, where said docking means comprises at least one zone with sealing means preventing leakage to occur.

3. The module as recited in claim 1, where said docking means comprises at least one hydrophobic zone preventing leakage to occur.

4. The module as recited in claim 1, where said docking means comprises at least one polymer film preventing leakage to occur.

5. The module as recited in claim 1, where said docking means comprises at least one o-ring, preventing leakage to occur.

6. The module as recited in claim 1, where said docking means comprises a sealant mechanism formed by patterning a polymer using lithographic technique.

7. The module as recited in claim 1, where said docking means comprises a sealant mechanism comprising a hydrophobic break formed using surface modifying technology.

8. The module as recited in claim 1, where said adhering units comprise a porous bed being kept inside each channel by restraining means.

9. The module as recited in claim 1, where said adhering units comprise at least a part of the defining surfaces of the channels and said defining surfaces can comprise surface modified silicon and/or porous silicon.

10. The module as recited in claim 1 further comprising a dispenser having a dispenser nozzle, a basin, a flexible membrane and a piezoelectric element, said element being arranged to controllably actuate the membrane and thereby cause the dispensing of a precise amount of a liquid residing in the basin.

11. The module as recited in claim 1 further comprising electrospray nozzles arranged in fluid communication with said channels, making said module compatible with tandem mass spectrometry using electrospray; or other type of ionisation.

12. The module as recited in claim 10 wherein the dividing walls have been partly or fully removed downstream restraining means, forming a common basin which all the channels are flowing into.

13. The module as recited in claim 10 wherein said dividing walls extend all the way to said dispenser thereby mechanically separating the flows from each channel.

14. The module as recited in claim 1 further comprising a free flow electrophoresis unit capable of generating an electric field, for separating the incoming solution into fractions containing biomolecules of different pH, where each of said channels is arranged in relation to the electric field for receiving a corresponding fraction of the solution from a parallel laminar flow.

15. The module as recited in claim 14 where said electrophoresis unit comprises a pair of electrodes integrated in the walls of the channel for generating said electrical field.

16. The module according to claim 1 and capable of handling analytes flowing continuously or non-continuously in one flow direction only.

17. The module as recited in claim 14 where said electrophoresis unit comprises a pair of electrodes arranged in side compartments having fluid connection to an isoelectric focusing compartment.

18. A module according to claim 1 for use as a storage unit, capable of retaining protein samples adhering to said adhering unit for long term storing at minus 20 degrees Celcius.

19. A method for processing biological specimens with increased speed comprising the following steps:
docking a dockable microextractor device, as recited in claim 1, to a priming device for loading microbeads into the extractor and flushing the extractor with a priming solution;
undocking the extractor from the priming device;
docking the extractor to a washing device;
flushing the extractor;
undocking the extractor from the washing device;
flowing a biological specimen in fluid phase through the dockable microextractor;
letting certain biomolecules adhere to units inside said extractor;
docking the extractor to an elution device;
eluting the certain biomolecules from the extractor;

20. The method as recited in claim 19 further comprising docking the extractor to a post extraction device

21. The method as recited in claim 20 where said post extraction device is a dispensing device

22. The method as recited in claim 21 where said dispensing device is of piezoelectric, mechanical, thermal resistor or electrospray type.

23. The method as recited in claims claim 19 where said flowing takes place in one flow direction only.

24. The method as recited in claim 23 further comprising the step of linearly moving a stage comprising a number of microextraction arrays.

25. The method as recited in claim 24 comprising the step of lifting and moving sideways one of said arrays.

26. The method as recited in claim 25 wherein said lifting is accomplished by vacuum picker means movable in two dimensions.

27. The method as recited in claim 26 comprising the step of vacuum sealing with a vacuum seal around the dispenser nozzle during washing of the dispenser.

28. The method as recited in claim 24 where the number of monolithic extraction arrays on the stage is 8.

29. The method as recited in claim 24 where the number of extractors in each array is 12.
